(19) United States
(12) Patent Application Publication
(10) Pub. No.: US 2018/0108442 A1
(43) Pub. Date: Apr. 19, 2018

(54) TELEMEDICINE REFERRAL PLATFORM

(71) Applicant: iDoc24 Inc., Berkeley, CA (US)

(72) Inventor: Alexander Börve, San Francisco, CA (US)

(73) Assignee: iDoc24 Inc., Berkeley, CA (US)

(21) Appl. No.: 15/787,460

(22) Filed: Oct. 18, 2017

Related U.S. Application Data
(60) Provisional application No. 62/409,621, filed on Oct. 18, 2016.

(51) Int. Cl. G16H 50/70; G16H 40/20; G16H 80/00; G16H 50/20

(52) U.S. Cl. CPC G16H 50/70; G16H 40/20; G16H 70/20; G16H 50/20; G16H 80/00

(57) ABSTRACT

A method for telemedicine referral implemented on a computer is provided. The computer includes a server and a data storage device in communication with the server. The data storage device includes a database of information for each of a plurality of dermatologists stored thereon. The method includes receiving, by the server, a case information package from a patient-facing computing device; The method further includes selecting, by the server, at least one dermatologist from the plurality of dermatologists to be provided with at least a portion of the case information package; transmitting, by the server, at least a portion of the case information package to a dermatologist-facing computing device corresponding to each of the selected at least one dermatologist; and receiving, by the server, from the dermatologist-facing computing device, assessment information of the skin condition; and transmitting, by the server, the assessment information to the patient-facing computing device.

### CROSS REFERENCE TO RELATED APPLICATION

This application claims priority to U.S. Provisional Patent Application No. 62/409,621 filed Oct. 18, 2016, which is hereby incorporated by reference herein in its entirety.

### TECHNICAL FIELD

Embodiments of the present disclosure relate to remote medical diagnosis and more specifically to diagnosis and triaging of animals or humans with skin conditions using mobile devices.

### BACKGROUND

Conventional systems for virtual medical consultation uses text messages, video chat, email, and/or portal applications. These interfaces often require a relationship to be established between the patient and medical professional. Establishing communication using these existing techniques can be expensive, inconvenient, and time consuming. And often once communication is established, the appointment may last minutes only for a patient to receive the message “come back if it gets worse.” Conventional interfaces can also be computationally inefficient at the medical professional-facing device. For example, traditional interfaces often require appointment scheduling, establishing a communication link, and/or other inefficient operations. A convenient and computationally efficient platform for medical diagnosis would therefore be useful.

### SUMMARY

An example method for telemedicine referral implemented on a computer is provided according to one embodiment. The computer includes a server and a data storage device in communication with the server. The data storage device includes a database of information for each of a plurality of dermatologists stored thereon. The method includes receiving, by the server, a case information package from a patient-facing computing device. The case information package includes at least one image of a skin condition of a patient. The method further includes selecting, by the server, at least one dermatologist from the plurality of dermatologists to be provided with at least a portion of the case information package; transmitting, by the server, at least a portion of the case information package to a dermatologist-facing computing device corresponding to each of the selected at least one dermatologist; and receiving, by the server, from the dermatologist-facing computing device, assessment information of the skin condition. The assessment information is determined by the selected at least one dermatologist based on, at least, the at least said portion of the case information package. The method also includes transmitting, by the server, the assessment information to the patient-facing computing device.

An example system for telemedicine referral is provided according to one embodiment. The system includes a server, and a data storage device in communication with the server. The data storage device includes a database of information for each of a plurality of dermatologists stored thereon. The server is configured to receive a case information package from a patient-facing computing device. The case information package includes at least one image of a skin condition of a patient. The server is further configured to select at least one dermatologist from the plurality of dermatologists to be provided with at least a portion of the case information package; transmit at least a portion of the case information package to a dermatologist-facing computing device corresponding to each of the selected at least one dermatologist; and receive from the dermatologist-facing computing device, assessment information of the skin condition. The assessment information is determined by the selected at least one dermatologist based on, at least, the at least said portion of the case information package. The server is also configured to transmit the assessment information to the patient-facing computing device.

An example non-transitory computer-readable medium having instructions stored thereon is provided. The instructions, when executed, cause a computer to perform a method for telemedicine referral. The computer includes a server and a data storage device in communication with the server. The data storage device includes a database of information for each of a plurality of dermatologists stored thereon. The method includes receiving, by the server, a case information package from a patient-facing computing device. The case information package includes at least one image of a skin condition of a patient. The method further includes selecting, by the server, at least one dermatologist from the plurality of dermatologists to be provided with at least a portion of the case information package; transmitting, by the server, at least a portion of the case information package to a dermatologist-facing computing device corresponding to each of the selected at least one dermatologist; and receiving, by the server, from the dermatologist-facing computing device, assessment information of the skin condition. The assessment information is determined by the selected at least one dermatologist based on, at least, the at least said portion of the case information package. The method also includes transmitting, by the server, the assessment information to the patient-facing computing device.

### BRIEF DESCRIPTION OF DRAWINGS

The foregoing and other features and advantages of the invention will be apparent from the following, more particular description of various embodiments, as illustrated in the accompanying drawings wherein like reference numbers generally indicate identical, functionally similar, and/or structurally similar elements. The first digits in the reference number indicate the drawing in which an element first appears.

FIGS. 1-7 depict example interfaces for a patient to input information associated with a medical condition and receive diagnosis and recommendation information from a medical professional registered with the platform.

FIG. 8 depicts an example interface for a patient to share information received from the platform.

FIGS. 9-12 depict example interfaces to generate a patient self-referral based on information received from the platform.

FIG. 13 depicts information from a medical professional associated with the platform that can be included in the patient self-referral.

FIGS. 14-16 depict the process of generating registration codes for a patient's device.

FIGS. 17-24 depict interfaces to register and activate a patient device with the platform.

FIG. 25 depicts a block diagram of a system to provide telemedicine referrals.

FIG. 26 depicts an interface for a doctor to submit case information to the platform on behalf of a patient.

FIGS. 27-38 depict a medical professional-facing application interface.

FIG. 39 depicts a web application interface for a patient to upload case information.

FIG. 40 depicts a personalized web application for an affiliate of the platform.

FIG. 41 depicts a schematic diagram of a telemedicine referral platform facilitated with artificial intelligence (AI) technology.

FIG. 42 depicts an example diagnosis of a skin condition that is facilitated with AI technology.

FIG. 43 depicts an example preliminary AI result for classifying a skin condition.

FIG. 44 depicts a flow chart of a method for a telemedicine referral platform.

FIG. 45 depicts an example computer system that may be used for a telemedicine referral platform.

### DETAILED DESCRIPTION

Illustrative embodiments are discussed in detail below. While specific embodiments are discussed, it should be understood that this is done for illustration purposes only. In describing and illustrating the embodiments, specific terminology is employed for the sake of clarity. However, the embodiments are not intended to be limited to the specific terminology so selected. A person skilled in the relevant art will recognize that other components and configurations may be used without departing from the spirit and scope of the embodiments. It is to be understood that each specific element includes all technical equivalents that operate in a similar manner to accomplish a similar purpose. The examples and embodiments described herein are non-limiting examples.

As used herein, the term “a” refers to one or more. The terms “including,” “for example,” “such as,” “e.g.,” “may be” and the like, are meant to include, but not be limited to, the listed examples. The term “product” may refer to both products and services.

Disclosed herein is a platform for medical consultation using a mobile device, such as a smartphone, tablet, and/or other mobile computing device. The techniques disclosed herein fill a gap in the medical information market by providing fast, personalized and professional medical information online. In certain cases, users of the platform with skin concerns may not have to step foot inside a clinic—they can self-treat with recommendations from specialists on the platform. In some cases, a specialist consulting a patient on the platform may send the user to a clinic via a self-referral. In certain cases, this referral process may be referred to as mobile teledermoscopy. Teledermoscopy (TD) may include a method that allows the diagnosis of skin diseases with the help of online clinical and dermoscopy photographs sent to a dermatologist for advice. The techniques disclosed herein streamline the treatment of skin related diseases, which may yield significant benefits. For example, 20% of all family doctor visits are skin related. There are 170 million skin-related doctor visits in the USA per year and 12,000 people die of skin cancer every year. 95% of these people could have been saved by early detection. The techniques disclosed herein may help to reduce the number of people afflicted with skin diseases.

In various embodiments, the techniques disclosed herein may be implemented as an anonymous healthcare information service. Through the platform, a user may be able to receive medical advice as to whether skin lesions, such as a rash, eczema and/or a visual change on your body constitutes a medical concern. The platform allows a user to determine whether to contact a specialist (dermatologist), wait, do nothing, and/or begin a specific treatment. The techniques disclosed herein offer first-hand medical specialist information and a possible diagnosis as well as triaging the right patient to the right doctor at the right time.

In various embodiments, an application is configured to capture one or more images (e.g., two images (one clinical and one dermascopic)), text with relevant clinical information about the patient history, the suspected condition (e.g., a suspected tumor), and/or other information related to the condition. In certain cases, hardware, such as a dermatoscope (e.g., a device including a magnifying glass with a light emitting diode (LED) configured to capture medical grade images), may be appended to the mobile device to aid in capturing clear images of the skin condition. For example, the dermatoscope may be attached to the camera of the mobile device and may enable the user to magnify a skin condition and capture a non-blurry image of the condition.

According to various embodiments, the images of the condition, text with relevant information about patient history, the suspected condition, and/or any other information from the patient may is packaged into a case information package (e.g., query package). The case information package may be provided to a server associated with the platform. In certain cases, the information may be anonymous, with no personal data (such as names, social security number, and/or other personally identifiable information). In certain cases, each submitted case information package may be assigned a unique case identifier (e.g., an eight-digit case number) linked to the patient/case. The case information packages may be received by an Internet platform (e.g., a Tele-Dermis® platform) on a dedicated server. A first network, a first computing device, a second network, a second computing device.

According to some embodiments, the case information packages (e.g., images of the skin condition and associated patient information) are securely provided from the mobile device associated with the patient to a server. The server receives the case information package, stores the case information package, and/or processes the packages for distribution to one or more medical professionals associated with the platform. In certain cases, the images of the skin condition included in the case information package may be processed to, for example, focus the images, anonymize the patient (e.g., by blurring the patient's face, blurring eyes, cropping the photo, etc.), and/or otherwise prepare the images for transmission to a medical professional. In some cases, the case information packages are organized in a queue for transmission to one or more medical professionals. In certain cases, the case information packages from various patients may be queued chronologically, based on severity, based on type of condition, and/or other parameters.

In some embodiments, a case information package is provided to an application and/or interface associated with a medical professional, such as a dermatologist. In certain cases, the case information package is provided to a mobile device associated with a medical professional. An image of the skin condition may be presented to the medical professional, and the medical professional may be presented an interface allowing them to either accept the case or reject the case. In certain cases, the medical professional may be presented an image of the skin condition (e.g., a thumbnail image, smaller image, etc.) and/or other information associated with the case (e.g., a short summary of the case). The medical professional may then be able to swipe in one direction to accept the case or swipe in an opposite direction to reject the case. In another example, the medical professional may provide alternate types of input to accept or reject a case. A medical professional may be provided multiple cases (from multiple patients) in succession until, for example, the medical professional decides to accept a case. Once the medical professional accepts a case, additional information from the case information package is displayed.

In various embodiments, the medical professional may evaluate the images of the skin condition, ailment description, and/or associated patient-related information in the case information package. Based on the assessment of the case information package, the medical professional may state a suspected diagnosis, suggestions for treatment (if necessary), input a priority level (e.g., priority 1-3), and/or provide other information. For example, the medical professional may provide a description of the clinical and/or dermascopical findings leading to the specified diagnosis. In certain cases, the case information package (e.g., TD referral) receives a reply within a set period of times, such as, but not limited to, 24-48 hours. The medical professional reply is provided to the server and provided to the application associated with the patient.

In some embodiments, no personal information is passed between the patient and medical professional. The case is only identified using the case identifier in both the patient-facing application and medical professional facing application. In other cases, personal information, may be securely passed between the patient and medical professional using the platform. The personal information (e.g., included in a case information package) may be encrypted at, for example, the patient's mobile device and/or the medical professional's application. The data sent directly between patient mobile device and medical professional interface may, for example, be secured using any suitable security mechanism, such as the secure sockets layer (SSL) communication protocol.

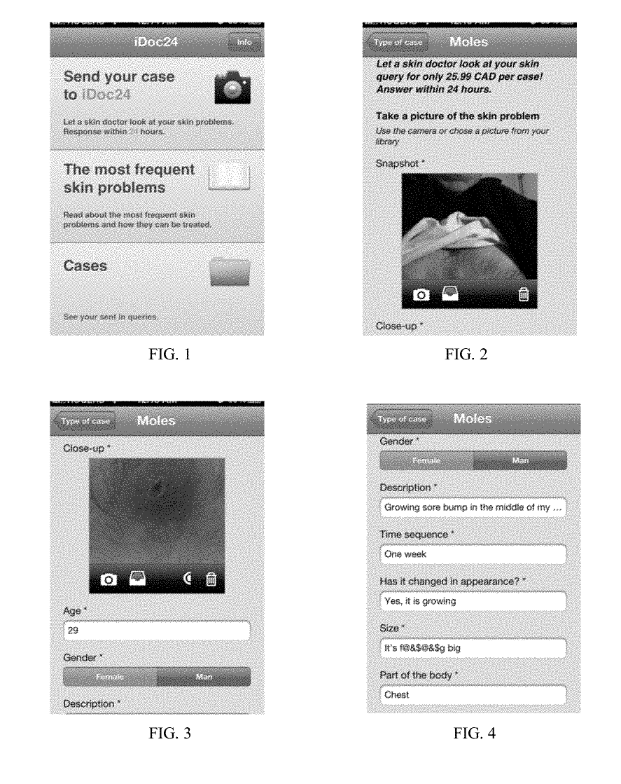

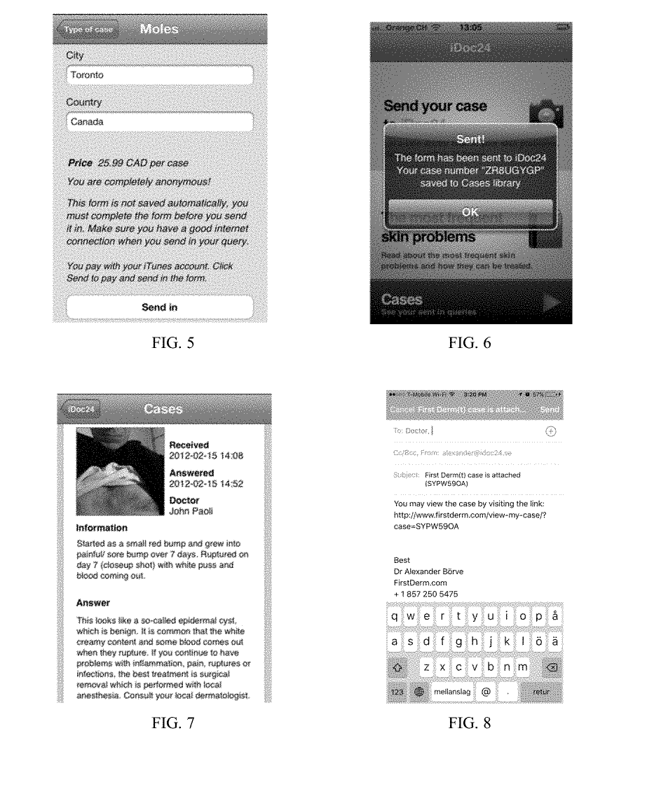

FIGS. 1-7 depict example interfaces for a patient to input information associated with a medical condition and receive diagnosis and recommendation information from a medical professional registered with the platform. FIG. 1 includes an opening page of a patient facing application. FIGS. 2-5 depict one or more interfaces for a patient to provide input information regarding a medical condition, such as a skin condition. FIG. 2 depicts an interface to capture images and/or input images of a medical condition. FIG. 3 depicts an interface for a patient to provide personal information. FIG. 4 depicts an interface for a patient to provide a description of the medical condition. The patient may, for example, provide a general description of the condition, a time period over which the condition has developed, a description of how the condition has changed, a location on the patient's body where the condition is located, and/or any other information associated with the condition. The images of the condition, patient information, information associated with the condition, and/or other information may be included in a case information package sent to a server associated with the platform. FIG. 5 depicts an interface for the patient to submit information associated with the condition to the platform. The patient may, for example, agree to pay a fee and press a “submit,” “send in,” or similar button. FIG. 6 depicts a dialogue confirming that the case information package has been sent to the platform. In certain cases, the case information included in a package is sent to a server. The server then assigns a unique identifier (e.g., an eight-digit/letter case number) to the case information package. The unique identifier may be used to retrieve the case information package without revealing or requiring patient confidential information. FIG. 7 depicts an interface in which a patient receives information from a medical profession regarding their condition. The interface may, for example, provide a name of the physician, a date submitted, a date of diagnosis, medical advice on how to remedy the condition (e.g., let it heal, see a specialist, go to the emergency room, etc.), and/or other information.

FIG. 8 depicts an interface for a patient to share the information received from the platform. In the example shown, a patient may share a link to information received from a medical professional on the platform. The information can be shared via email, text message, and/or other communication approach.

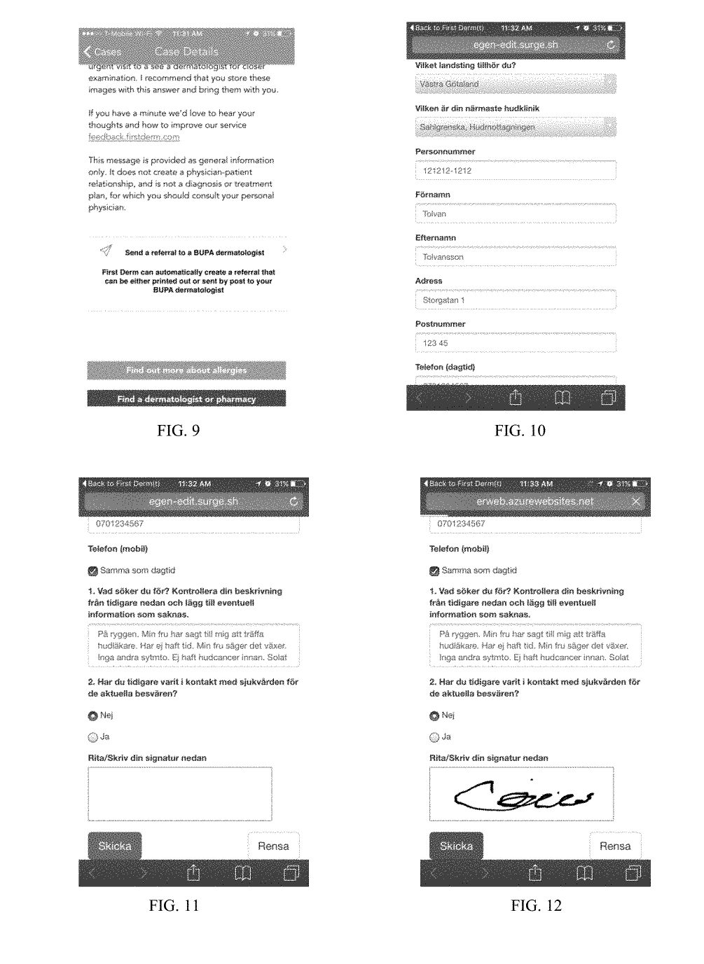

FIGS. 9-12 depict interfaces to generate a patient self-referral based on information received from the platform. FIG. 9 depicts an interface to generate a self-referral for the patient. The self-referral may be based on the information received from the medical professional using the platform. In certain cases, the patient self-referral interfaces may be displayed to a user only when a medical professional recommends that the patient see a specialist regarding their condition. The self-referral interface may assist a patient in finding a specialist, pharmacy, or other medically-related entity to remedy the condition. FIG. 10 depicts an interface for the patient to provide their personal information (e.g., contact information) and select a specialist. FIGS. 11-12 depicts an interface for a patient to provide further personal information. For example, the user may provide further information on their condition, information on any previous medical consultations, and/or other information. The patient may also provide a signature.

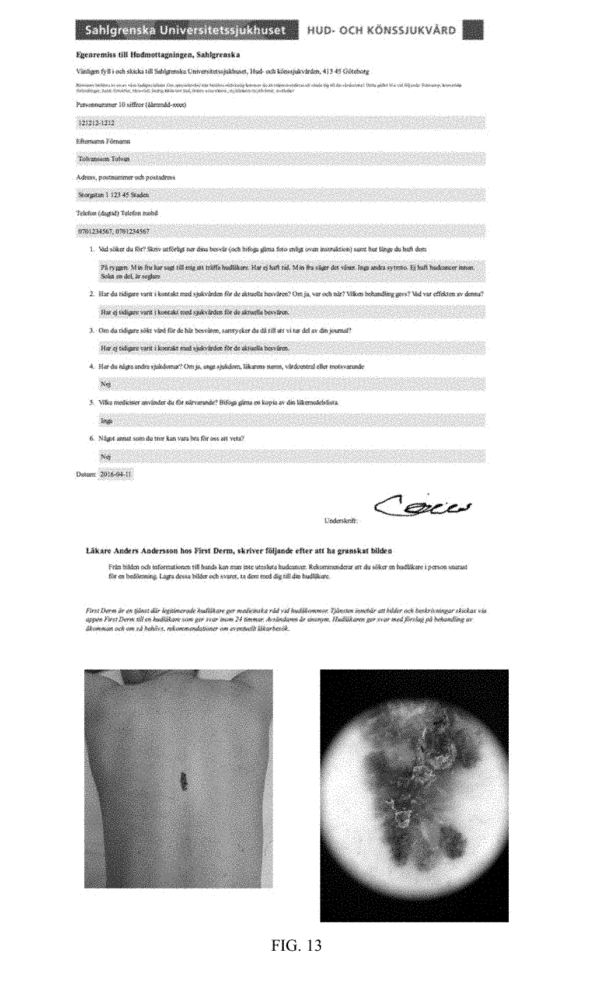

FIG. 13 depicts information from a medical professional associated with the platform that can be included in the patient self-referral. For example, the referral can include the answer from dermatologist associated with the platform, images that the patient provided to the platform, and/or other information. This information can be provided to a portal associated with the specialist to which the patient has been self-referred. In certain cases, the information may be automatically mailed to the referral clinic, sent by email, or otherwise transmitted. The clinic to which the patient is referred may then contact patient and organize a visit.

In some embodiments, when a patient needs to see a dermatologist based on the information received from the platform (e.g., the skin condition is diagnosed as skin cancer, the patient may press a self-referral button on the patient-facing computing device, for example to connect with a closest dermatologist clinic or choose which clinic. The patient may further press a “send” button. A self-referral case is generated that may include a dermatologist's answer and may be sent by e-mail, via a connection with EMR, or snail mail. The patient may get a copy of the self-referral case that has been sent and may follow up themselves (empowering the patient with their own user data).

For example, the patient may view a case by entering a case number (e.g., E7AP0S0W) on the patient-facing computing device and then view the answer to this case by a medical professional. The patient may press a ZocDoc link (it can also be a link to partner dermatologist website (EMR—booking system) or nearest dermatology clinic). The patient is connected with a dermatologist, books an appointment and adds their First Derm answer for the dermatologist to review. It could alternatively be sent by conventional physical mail to clinic. The patient may get a confirmation or a copy of the postal mail. The patient may further need to follow up on the appointment (empowerment).

In some embodiment, when the answer is given to the patient user, there is a possibility in the answer to send the answer as a “self-referral” either electronically or by normal mail. The user can force an in-person medical appointment by sending the answer given by the dermatologist as a “self-referral”. The receiving entity will act on the urgency of the “self-referral”, to justify an in-person visit and whether it should be prioritized in regards to the severeness of the skin disease (e.g., malignant melanoma).

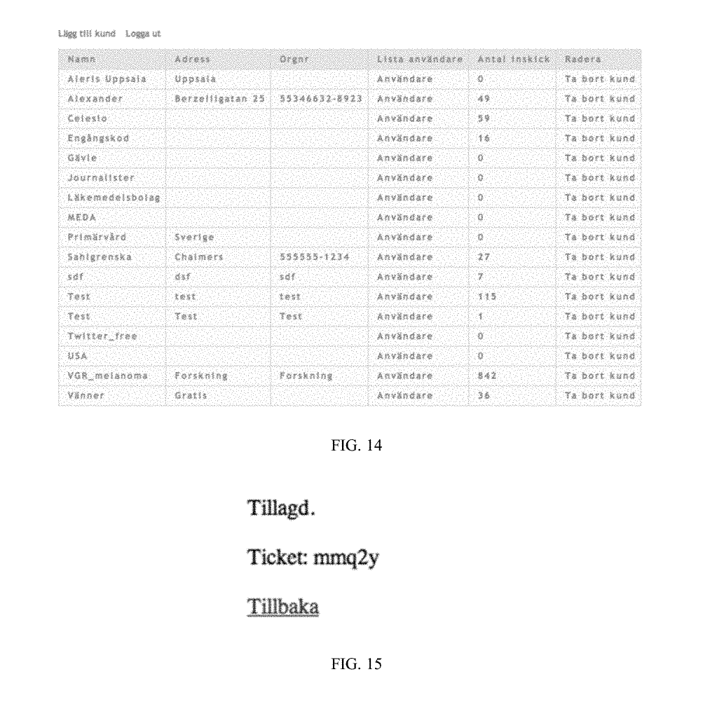

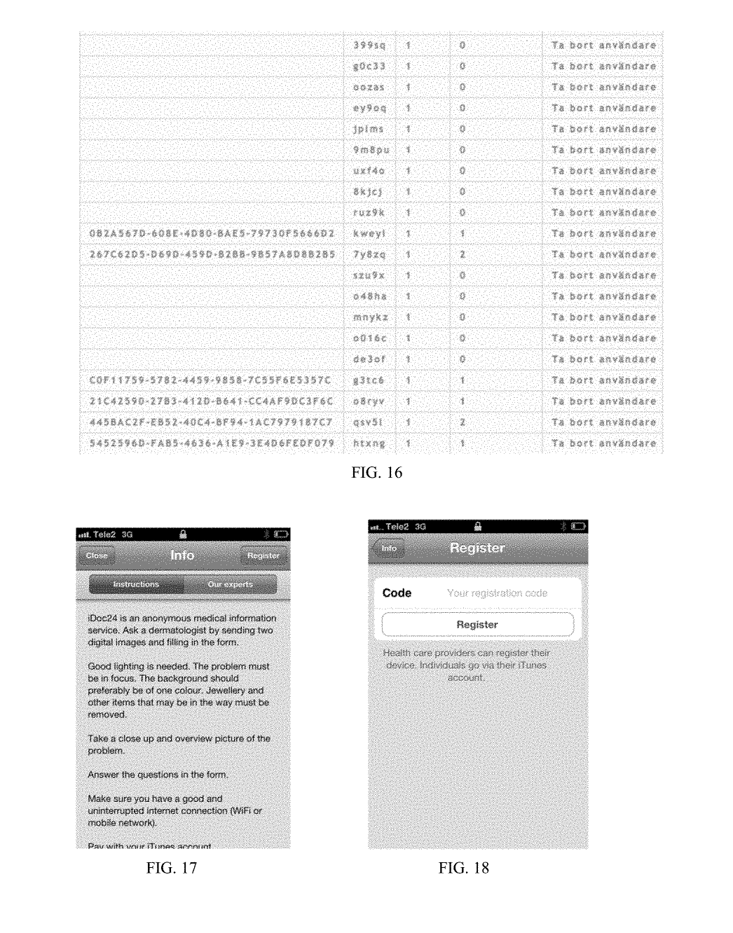

FIG. 14-16 depict the process of generating registration codes for a patient's device. In some embodiments, registration codes are generated for patient's devices. The registration codes may be generated at a server associated with the platform. FIG. 14 depicts a database of users on the platform. FIG. 15 depicts a random code generated for a patient user of the platform. In the example shown, a patient user is provided a randomly generated registration code “mmq2k.” This code can be used to register the patient user's application and/or mobile devices with the platform. FIG. 16 depicts a database of registration codes correlated to specific users. The codes may be confirmed to a specific patent device.

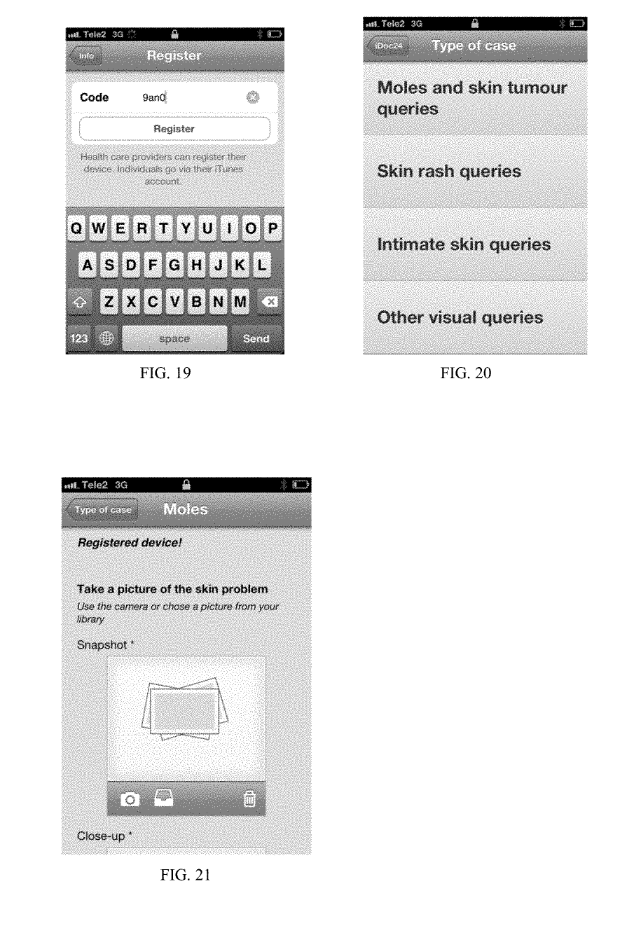

FIGS. 17-24 depict interfaces to register and activate a patient device with the platform. FIG. 17 depicts a registration interface including a “register” button. FIGS. 18-19 depict an interface where a patient user and/or a health care provider may enter a registration code. FIG. 20 depicts an interface in a patient user application allowing a patient user to select a form to provide information regarding a skin condition. In the example shown, a user may select a form for “Moles and Skin Tumor Queries,” “Skin Rash Queries,” “Intimate Skin Queries,” and/or other “Other Visual Queries.” In certain cases, the list of forms presented to a user may depend on the registration code entered by the user. For example, different registration codes may unlock different lists of forms.

In various embodiments, registration codes associated with the application can “activate” hidden forms. The hidden form may change where the queries are sent (e.g., to a specific medical professional associated with platform). For example, a hidden form may specify uniform resource locator (URL) where the query may be sent. In certain cases, the content of the forms output to the patient user can be changed. For example, the content of the forms can be used for research purposes, and the hidden forms may include different prompts for different patient users.

FIG. 21 depicts an interface for a patient user to provide information regarding a medical condition, such as a skin condition. Similar to the interfaces depicted in FIGS. 1-7, a patient user may input information regarding a skin condition. The patient's query can be transmitted directly to the platform (e.g., for analysis by a medical professional).

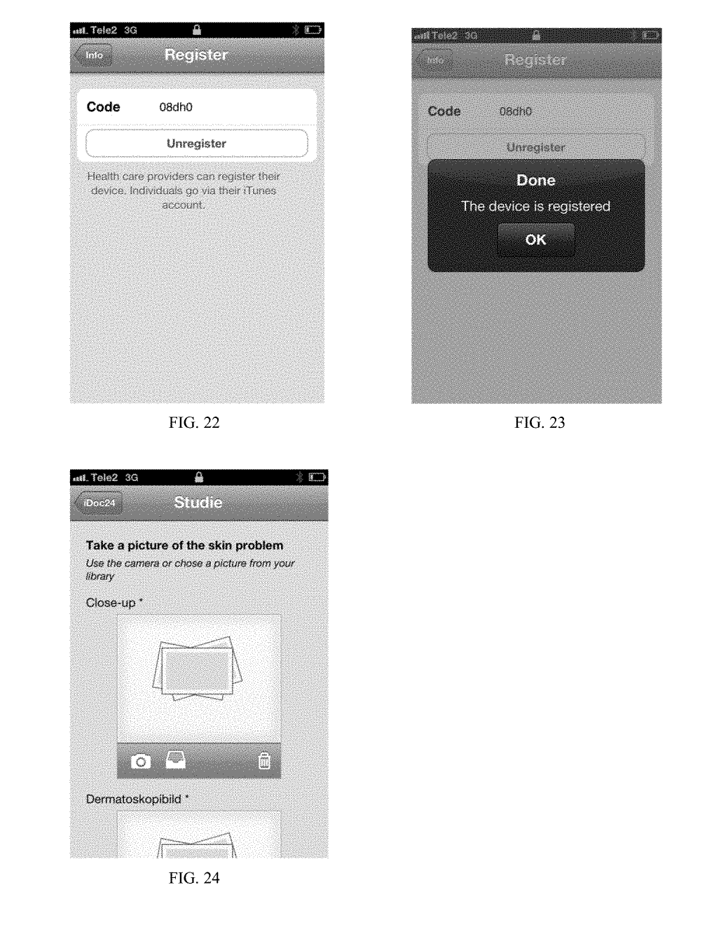

FIG. 22 depicts an interface to change a registration of a patient user application. For example, the platform may be used by a general practice medical facility, and a physician may enter a code associated with a patient to activate the application for that patient. An instance of the application can be activated for different users of the platform. This allows confidentiality to be maintained for each given user, and allows the platform to provide a custom interface for each patient user.

FIG. 23 depicts an interface indicating that the device is registered to an account associated with a particular registration code.

FIG. 24 depicts an alternate interface accessed based on a particular registration code. In the example shown, an interface tailored to a medical study is provided based on a particular registration code. Other specific forms may be “hidden” in app and activated with a predefined registration number.

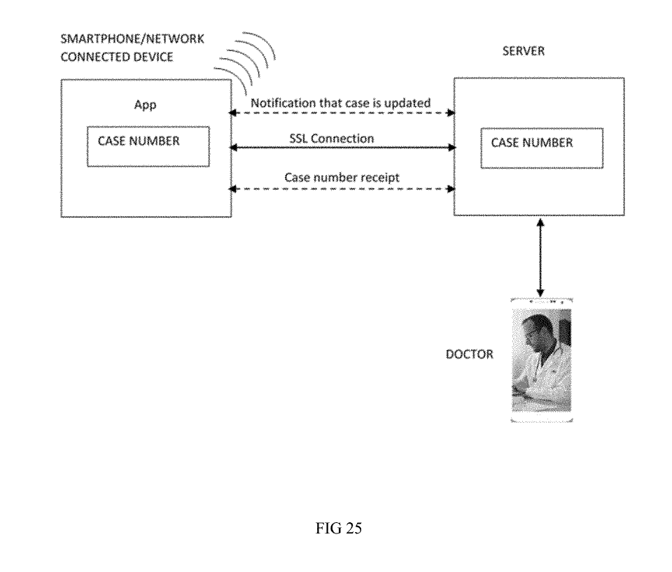

FIG. 25 depicts a block diagram of a system to provide telemedicine referrals. In the example shown, a mobile device (e.g., smartphone) associated with a patient may include an application associated with the platform. A secure connection (e.g., an SSL connection) is established between the application and a server associated with the platform. The user registers the application with the platform. The patient user then uses the application to document a skin condition by, for example, taking pictures of the condition, providing description information, and the like. A case information package is generated at the application including this information. For example, the case information package may be encrypted at the application. The case information package is provided to the server. The server generates a case identifier for the case package. The case number may include a random eight character identifier. The server may provide a receipt that the case has been uploaded. The server may also provide the case identifier to the patient's application. The patient and/or other parties authorized by the patient can use the case identifier to access information regarding the case, such as diagnosis/recommendation information from a medical professional associated with the platform. For example, a patient's physician may access the patient's case on their mobile application. In certain cases, the only way a user can access information regarding the case is using the case identifier. In such a scenario, no patient personally identifiable information (PII) (e.g., name, gender, email address, IP address, phone number, address, etc.) will be exposed on the platform, and PII cannot be used to access a case associated with the patient.

In various embodiments, FIG. 25 depicts the communications between a patient-facing application and server associated with the platform. A similar architecture can be deployed for communication between the medical professional-facing application and server. For example, the medical professional-facing application and/or desktop interface may securely communicate with the server in a manner similar to the patient-facing application.

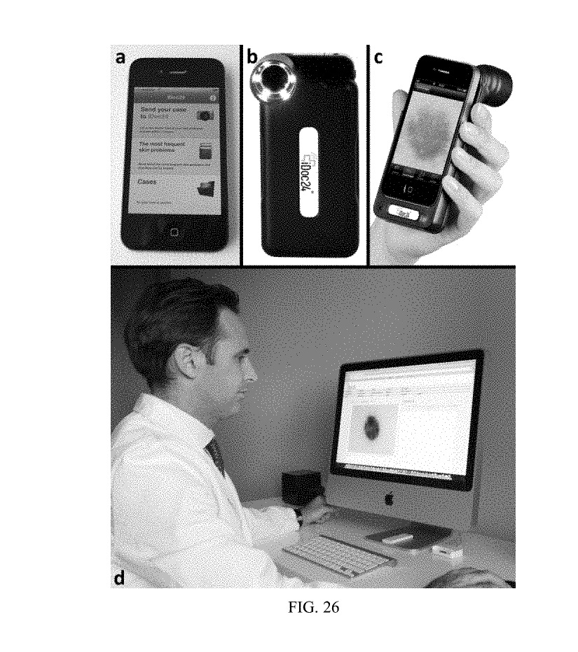

FIG. 26 depicts an interface for a doctor to submit case information to the platform on behalf of a patient. In certain cases, a dermatoscope (e.g., a device including a magnifying glass with a light emitting diode (LED) configured to capture medical grade images) may be used to capture images of a skin condition. For example, a general practitioner that does not specialize in skin disorders may capture images of a patient's skin condition, enter a description of the condition, and provide other information to the platform via a web interface. In this scenario, the physician may interact with the platform in a similar manner to a patient.

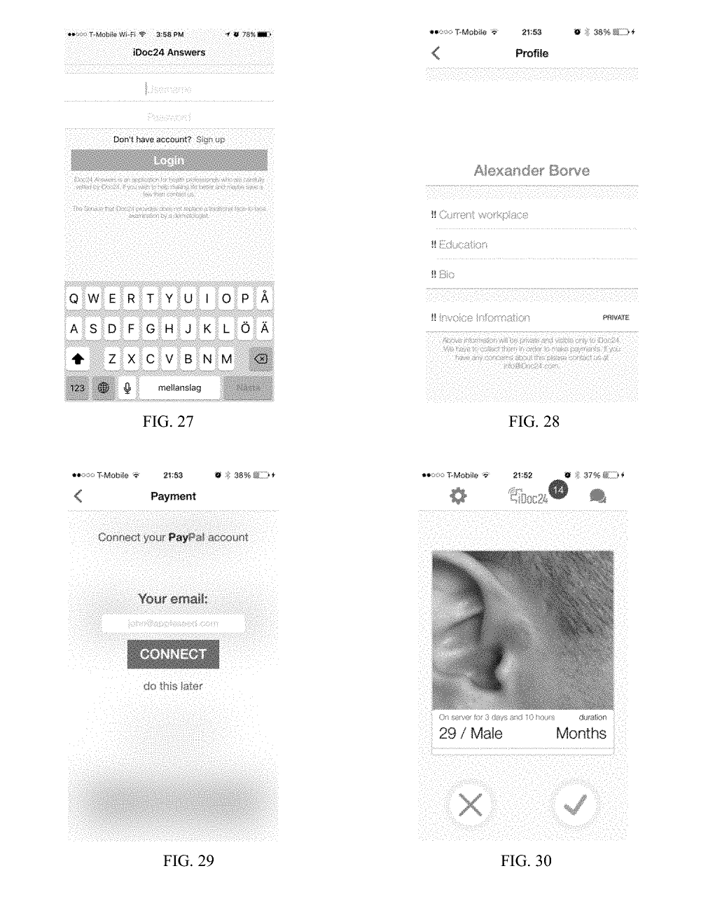

FIGS. 27-38 depict a medical professional-facing application interface. The medical professional-facing application (e.g., a dermatologist answering application) is accessible to a medical professional, such as a dermatologist, skin specialist, etc., that is registered with the platform. In certain cases, the medical professional-facing application can include a web application, a desktop software application, mobile device application (e.g., iOS application, Android application, etc.), and/or any other type of software. Using the application, the medical professional is presented with case information (e.g., queries) from multiple patients. In certain cases, an interface is provided for the medical professional to accept or reject a case. In one example, a medical professional can swipe left on cases they do not want to answer and swipe right on cases they do want to answer.

FIG. 27 depicts an interface for a medical professional user to register with the platform. A medical professional may for example register a username and password with the platform. The username and password may be used to login to the platform. In certain cases, dual-factor authentication may also be used.

FIG. 28 depicts an interface for a medical professional user to register with the platform. In the example shown, a medical professional user can provide their clinic information, educational credentials, biography, license information, billing information, and/or other information describing their practice. This information (or a portion thereof) may be provided to a patient along with a diagnosis.

FIG. 29 depicts an interface for a medical professional to register their payment account with the platform. For example, the medical professional can integrate an e-payments platform (e.g., PayPal, Braintree, etc.) with their account and receive automatic payment after evaluating a case. The medical professional (e.g., a dermatologist) can get paid on the go. For example, the medical professional could answer four cases, and can press a “cash out” button on the medical professional-facing computing device. An invoice, for example a connection to W2 form is generated, and their money is deposited in their Bank or paypal account that is connected.

Once the medical professional is registered with the platform, case information packages may be provided to the medical professional's application. In various embodiments, the server includes a queue of cases associated with various patients. The cases may be organized based on time since upload, potential type of disorder, location on the body, number of times the case has been rejected by physicians, severity, and/or any other parameters. In one example, case information may be held on the server for a period of time (e.g., 12 hours, a day, etc.) before being provided to a medical professional's device. In this case, cases are delayed at the server so the patient does not receive a result too quickly, which may create the impression that the medical professional made a hasty diagnosis. In another example, cases that have been rejected by one or more medical professionals may be advanced in the queue and/or have a higher priority. A case that has been rejected multiple times may remain near the top of the queue until it is accepted by a medical professional. By way of an additional example, the subject matter of a case, such as the type of condition, may be automatically evaluated, and this information may be used to determine a medical professional and/or set of medical professionals to receive the case. Using image recognition techniques, the server can provisionally classify the condition as a rash, potential tumor, and/or other condition. And based on the classification, the case may be queued for evaluation by a particular medical professional or set of medical professionals.

Processing this queue, case information may pushed or pulled to devices associated with medical professionals. In one example, a medical professional may log in to the application, and case information is pulled from the server to the medical professional's device. In another example, the server may push case information to various medical professional devices when, for example, the information is received at the server, when the case information is ready for diagnosis, and/or at other times.

FIG. 30 depicts an interface for a medical professional to accept or reject a case from a patient. In the example shown, the medical professional-facing application receives a case information package and displays information related to a particular patient's case. The case information may include images of the potential condition, an age of the patient, gender of the patient, a duration over which the patient has been afflicted with the condition, and/or other information. In certain cases, the medical professional is not provided any information regarding the identity of the patient at this point. For example, an image of the condition may be processed to blur out features (e.g., the patient's eyes, face, tattoos, etc.) that could identify the patient.

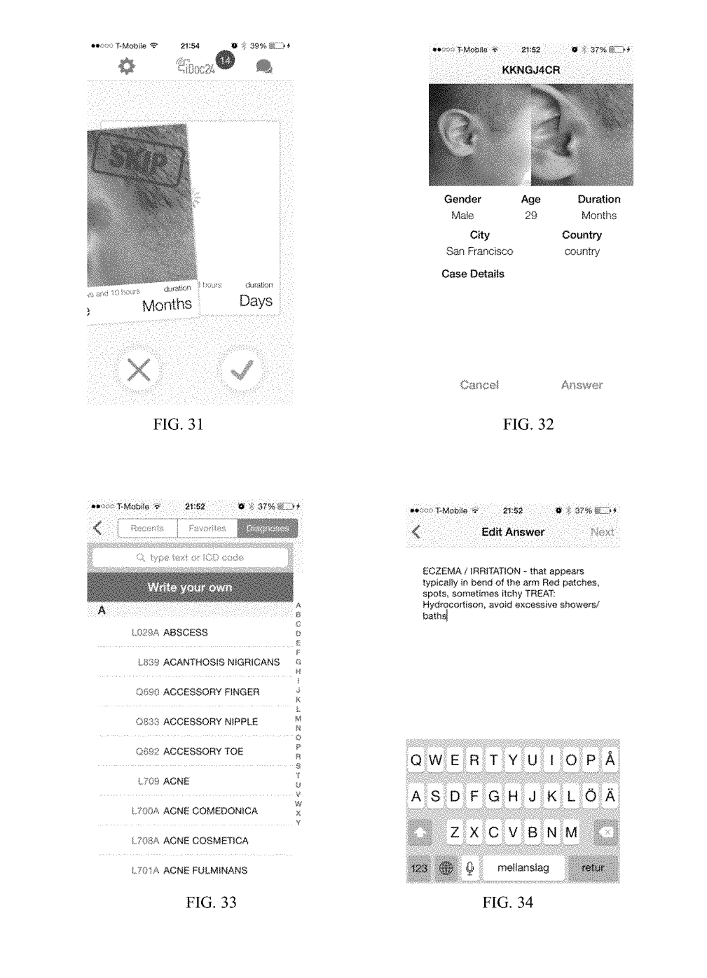

FIG. 31 depicts an interface for a medical professional user to reject a case. In the example shown, a medical professional may swipe to the left to reject a potential case. The medical professional user may, for example, determine that the condition is not within their realm of expertise and may pass on evaluating the case by swiping left. When the medical professional rejects the case, another case is provide in the application. Using this process, the medical professional may superficially evaluate multiple cases and select only the cases that they are confident in diagnosing.

In various embodiments, once a case is rejected by a particular medical professional, the case may be made available to other medical professionals. In certain instances, a case information package is updated when a case is rejected to indicate that the case has been rejected and/or to include the identity of the medical professional that rejected the case. A case that has been rejected one or more times may be prioritized (at the server) above a case that has not been evaluated by a medical professional. In some instances, an indication may be provided with a particular case that it has been rejected, for example, X number of times. Such an indication may incentivize a medical professional to accept the challenge of diagnosing the case (i.e., a case their colleague could not figure out). In some cases, an amount paid to a medical professional may be proportional to the number of times the case has been rejected, the length of time the case has been on the platform, and/or other factors. For example, if a case has been rejected by multiple professionals, it may be deemed a difficult case, and a medical professional that accepts the case may be paid more.

In some embodiments, each medical professional is associated with a rank and/or weight. Medical professional users can be ranked by the number of cases successfully answered, and the rank of a medical professional that rejects a case may affect the priority of that case. For example, a case that is rejected by a lower ranked medical professional may have a lower priority (e.g., location in the queue at the server) than a case rejected by a higher ranked medical professional.

FIG. 32 depicts an interface for a medical professional user to accept a case. In the example shown, a medical professional may swipe to the right to accept a potential case. Upon accepting a case, additional information may be output to the medical professional user. For example, an additional zoomed out image of the skin condition can be provided. Once the medical professional accepts the case, additional patient-provided information can also be output, such as case identifier code (e.g., an eight character code), the patient's description of the condition, the patient's location of residence, and/or other information. The medical professional-facing application may also allow the medical professional to zoom in, crop, filter, and/or otherwise process the image of the skin condition.

In some embodiments, when a medical professional (e.g., a dermatologist) looks at an image of a skin condition, the medical professional may swipe a filter on and get a probability reading of the skin condition, which may help him make the diagnosis of the skin condition. This filter can be a stand-alone filter and can be used on any picture/image application, where there is a lot of “skin”. Any user can swipe the “filter” and get a reading on their skin.

In various embodiments, when a medical professional user accepts a case, that case may be checked out to that medical professional. The case will not be presented to other medical professionals on the platform. In certain cases, the case will be checked out to the medical professional user for a specific period of time, such as 12 hours. In the event the medical professional does not complete evaluation within the period of time, the case may be returned to the queue and made available to other medical professional users.

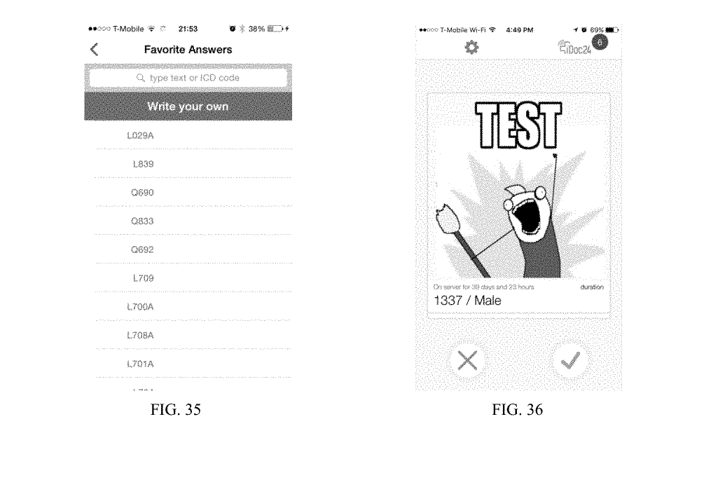

FIG. 33-36 depict interfaces for a medical professional user to provide a diagnosis and/or recommendations. As depicted in FIG. 33, a medical professional may select a diagnosis from a list of potential conditions. The list of potential conditions may be retrieved from, for example, the server associated with the platform, a third party data source via an API, and/or any other source. The list may be case searchable by keyword, diagnosis-related codes, and/or other information. As depicted in FIG. 34, a medical professional may provide a custom diagnosis and/or recommendation. In certain cases, the medical professional may edit a template diagnosis to include additional information. For example, the template diagnosis may include a description of the condition, but the medical professional can add a proposed treatment for the condition. As depicted in FIG. 35, a medical professional user may be store a list of favorite/frequent diagnoses. These favorites can be synchronized to the server (backend) associated with platform. In certain cases, favorite/frequent diagnoses can be shared among multiple medical professionals. Each of the diagnoses may be assigned a code, such as international classification of diseases (ICD) code. FIG. 36 depicts an interface displaying a next case after a medical professional user has completed diagnosis of a case. For example, after completing a case the medical professional facing application presents a next image to be evaluated. The medical professional can then swipe left (reject) or swipe right (accept) on the image.

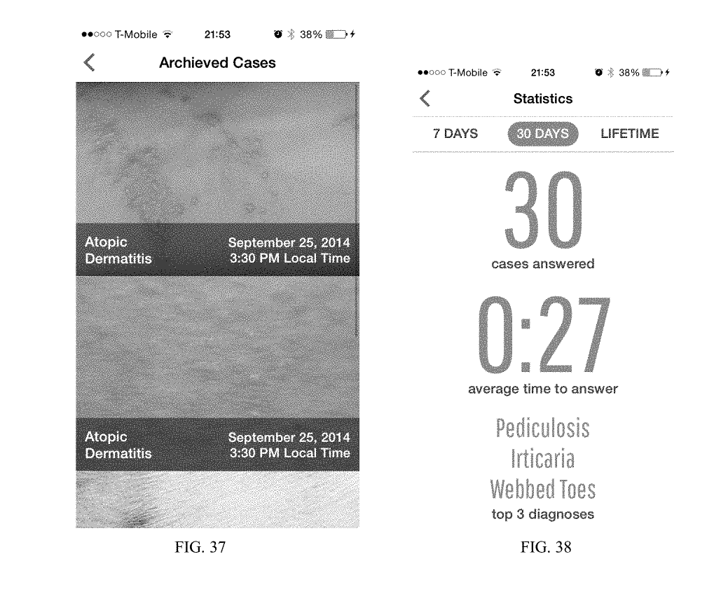

FIG. 37 depicts an interface displaying a medical professional's answered cases. In the example shown, a list of photographs and information for a medical professional's recently answered cases is presented. A medical professional may refer to this list in generating a diagnosis for a new case or may use the list to track their activities.

FIG. 38 depicts a display of a medical professional's diagnosis statistics. In the example shown, a number of cases answered, average answer time, top three diagnosed conditions, and/or other information are displayed. In certain cases, the most popular diagnosed conditions across the platform, within a set of medical professional users, and/or amongst another group is displayed.

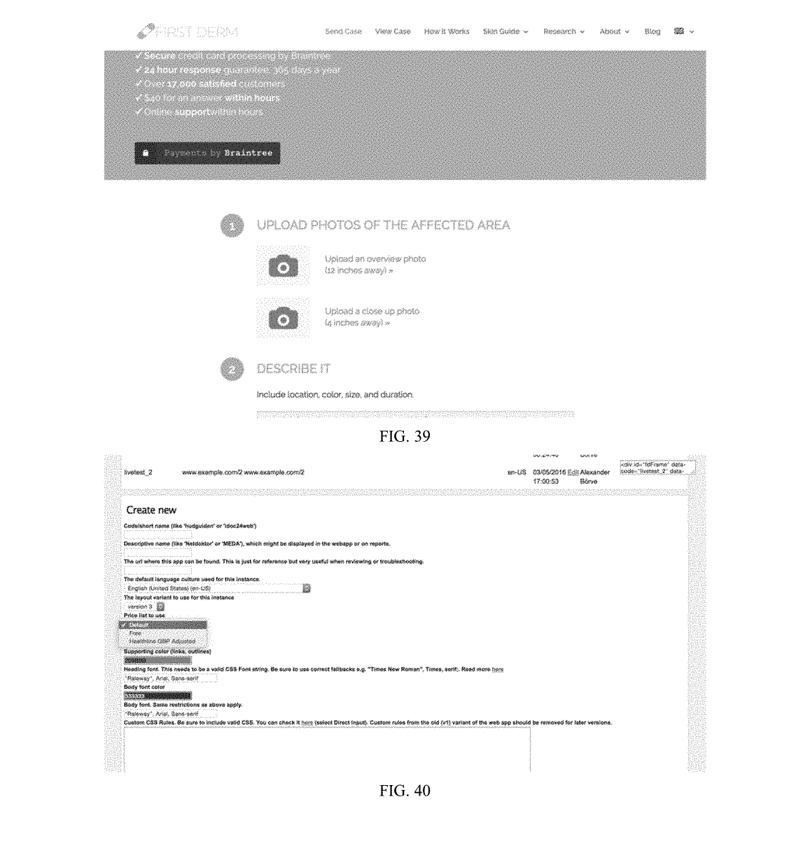

FIG. 39 depicts a web application interface for a patient to upload case information. The case information may be uploaded via web application (e.g., an iFrame) embedded on website. The web application may function similar to the mobile application interfaces described herein. Images of a skin condition, description of the condition, and/or other information are uploaded via the web application. A patient may also be able to pay via a payment system link embedded in the web application (e.g., a Braintree link, PayPal link, etc.). The patient may also receive a case number, look up diagnosis and/or recommendation information when a medical professional answers, and/or perform other operations via the web application.

FIG. 40 depicts a personalized web application for an affiliate of the platform. In the example shown, a health care provider (e.g., a general practitioner) associated with the platform may upload case information for diagnosis via a web application. In certain cases, an iFrame web application to a specific an affiliate (e.g., by URL and name) is presented. The web application (e.g., iFrame) is connected to a payment system (e.g., PayPal, Braintree, etc.) that incorporates revenue share. In certain cases, when a user used the service and pays. The affiliate gets paid directly to their payment system account after user have used our service depending on revenue share agreement. The affiliate may receive a percentage of the fee paid by the patient. The affiliate can embed the web application code (e.g., iFrame code) anywhere or even share via e-mail/text.

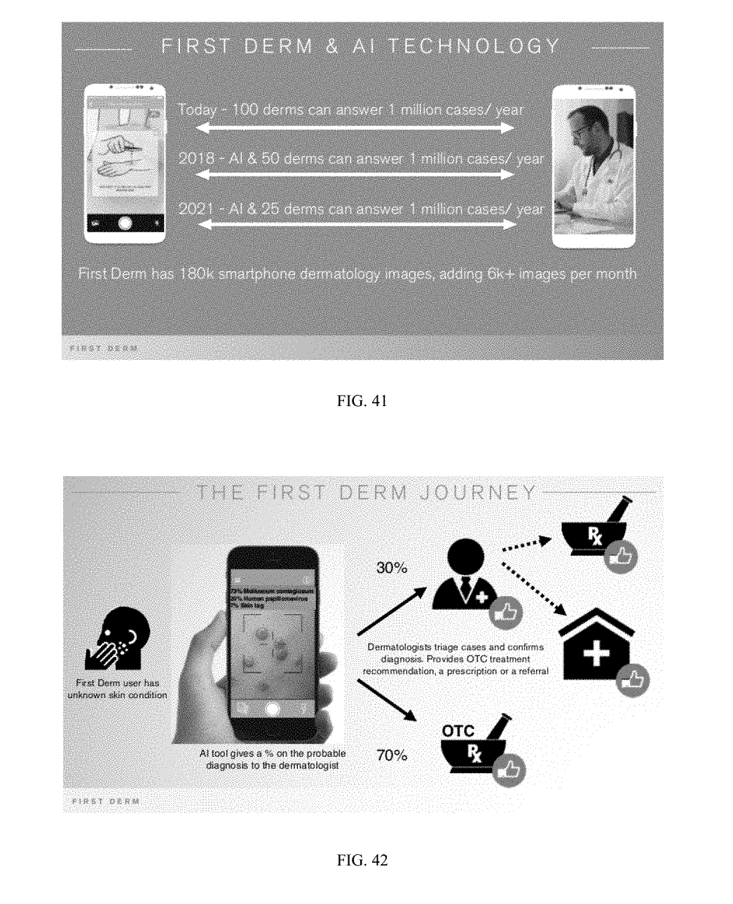

In some embodiments, artificial intelligence (AI) techniques may be used to facilitate diagnosis of skin conditions. As used herein, the AI techniques employed for a telemedicine referral platform may include, but are not limited to, machine learning, artificial neural networks, and computer vision. For example, the AI can be used at a server of the telemedicine referral platform for acquiring, processing, analyzing and understanding digital images of skin conditions received from patient-facing computing devices. As shown in FIG. 41, a variety of skin conditions images can be handled using AI techniques, which can significantly improve efficiency of the telemedicine referral platform, and reduce labor cost and resources requirements.

In one embodiment, pattern or image recognition can be used for the recognition of patterns and regularities in skin conditions digital image data to classify the skin conditions. Pattern recognition systems may be trained from labeled “training” data (e.g., supervised learning). When no labeled data are available, other algorithms may also be used to classify unknown skin condition patterns (i.e., unsupervised learning). Using image recognition techniques the server can provisionally classify a skin condition as a rash, potential tumor, and/or other condition. As shown in FIG. 42, a patient sends an unknown skin condition to the server where AI (e.g., image recognition) may be employed to analyze the unknown skin condition and to further classify it as one or more probable conditions. The results of classification may be relayed to a medical professional who uses a computing device (e.g., a smartphone or a computing tablet) to view the image of the unknown condition and the corresponding classification results. As shown in the FIG. 42, the unknown condition may be classified as 73% molluscum contagiosum, 20% human papillomavirus, and 7% skin tag. Such classifications can greatly assist the medical professional to diagnose the unknown conditions quickly, effectively, and accurately. The medical professional may triage the conditions and confirm diagnosis, and further provide over-the-counter (OTC) treatment recommendations, a prescription, and/or a referral. In addition, the resultant classification information may be used to determine a medical professional and/or a set of medical professionals to receive the case for further diagnosing the skin condition, for example based on the registered profile of the medical professional and/or previous cases received by the medical professional.

In some embodiments, the dermatologist-facing computing device may be configured to be a swiping filter to trigger the AI techniques to analyze the images of skin condition. For example, the swiping filter is included in an application installed on the dermatologist-facing computing device. When an image is swiped over, the AI is triggered to start analyzing the image to predict what it can be. For example, text may be prompted automatically on the screen (e.g., labeling the image) to indicate a probability of a specific type of skin condition that the image may be. For example, the skin condition on the image may be a probability of 90% as melanoma.

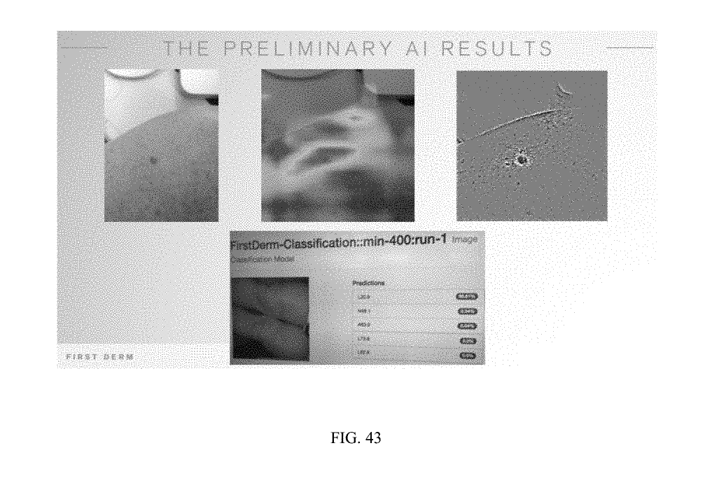

FIG. 43 illustrates an example preliminary AI result. After AI analysis at the server, the patient's skin condition is predicted as potential conditions listed in the FIG. 43, where the International Classification of Diseases (ICD) codes are used to label the diagnosis.

In some embodiments, a case may be evaluated by a plurality of medical professionals in a sequence or simultaneously. For example, when a medical professional receives the case, he may not be able to definitively determine the diagnosis of a skin condition. The medical profession may only offer a probable diagnosis and recommend further diagnosis by one or more other medical professionals. A case information package including the probable diagnosis may be returned to the server. The server may then transmit the case to other medical professional facing devices for further diagnosis. Also the medical professional may invite other medical professionals to diagnose the skin condition online at the same time.

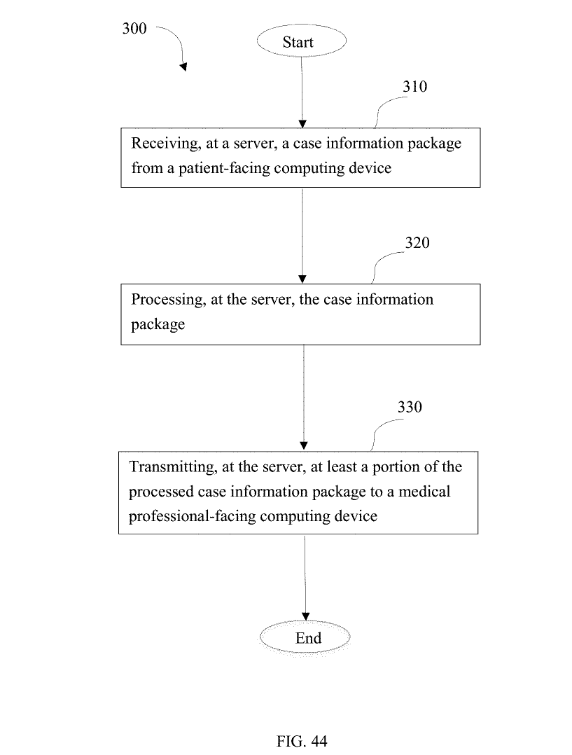

FIG. 44 shows a flow chart of a method 300 for using the telemedicine referral platform in accordance with one embodiment. The method 300 may be implemented in the telemedicine referral platform systems described herein, and may include one or more of the following steps.

At step 310, a case information package is received at a server of the telemedicine referral platform from a patient-facing computing device. The case information package may include one or more of: one or more images of a skin condition of a patient captured by the patient-facing computing device, information associated with the patient, a suspected condition of the skin condition, and a description of the skin condition. The description of the skin condition may include one or more of: a time period over which the skin condition has developed, how the skin condition has changed, or a location where the skin condition is located. The one or more images may also be taken by another device such as a dermatoscope, and uploaded into an image folder or library on the patient-facing computing device.

At step 320, the case information package may be preliminarily analyzed and processed at the server. For example, the one or more images may be classified using various AI techniques to predict a probable diagnosis for the skin condition shown in the images. The processing may include one or more of: predicting a probable diagnosis about the skin condition, focusing at least one of the images of the skin condition, anonymizing the information associated with the patient, and organizing the case information package in a queue. The queue may be organized based on one or more of: an order in which the case information package is received at the server, a number of times the case information package has been rejected at patient facing computing devices, a severity level of the skin condition, or a type of the skin condition.

At step 330, at least a portion of the processed case information package is transmitted at the server to a medical professional-facing computing device. The portion may include a thumbnail image of the one or more images in the case information package and/or a brief description of the skin condition without further revealing other information of the patient who submits the case information package.

After receiving the portion of the processed case information package, the medical professional who uses the medical professional-facing computing device may decide, based on the portion displayed on the medical professional-facing computing device and his expertise and experience, whether he would like to accept this case or decline this case.

If the portion of the processed case information package is accepted, additional information from the processed case information package may be displayed on the medical professional-facing computing device and presented to the medical professional for diagnosis of the skin condition.

Once being assessed, the case information package with the diagnosis information may be returned to the server. The assessing and diagnosis process may include one or more of: select a diagnosis about the skin condition from a list of potential conditions, state a suspected diagnosis of the skin condition, suggest a treatment for the skin condition, or input a priority level of the skin condition.

In some embodiments, the method 300 may further include receiving, at the server, from the medical professional-facing computing device the assessed case information package; and sending, from the server to the patient-facing computing device, the assessed case information package. The assessed case information package may include one or more of: a name of a medical professional who diagnosed the skin condition, a date on which the assessed case information package was submitted by the medical professional, a date on which the skin condition was diagnosed by the medical professional, or medical advice on how to remedy the skin condition.

In various embodiments, the method 300 may also including generating, at the server, a registration code for the patient-facing computing device; providing a list of forms, based on the registration code, to the patient-facing computing device for inputting the case information package; and registering a medical professional with the telemedicine platform. The registration may include one or more of: clinic information of the medical professional, educational credentials of the medical professional, biography of the medical professional, license information of the medical professional, and billing information of the medical professional

In some embodiments, the method 300 may further include updating, at the server, a priority of the case information package in the queue based on one or more of: a rank of a medical professional who rejects the case information package, a number of times the case information package has been rejected, and a period of time the case information package is held on the server.

The server and computing devices described above can embody a smart phone, a computing tablet, a desktop computer, a laptop computer, a mainframe, or the like.

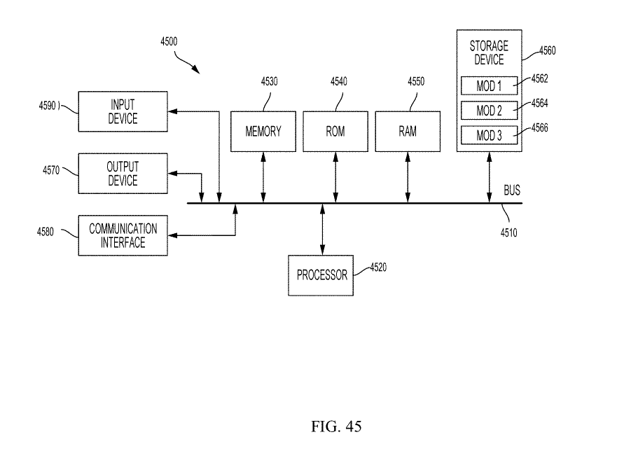

FIG. 45 illustrates an example computer system 4500 which can be used to perform the systems and methods for telemedicine referral platform as disclosed herein. The exemplary system 4500 can include a processing unit (CPU or processor) 4520 and a system bus 4510 that couples various system components including a system memory 4530 such as read only memory (ROM) 4540 and random access memory (RAM) 4550 to the processor 4520. The system 4500 can include a cache of high speed memory connected directly with, in close proximity to, or integrated as part of the processor 4520. The system 4500 copies data from the memory 4530 and/or a storage device 4560 to the cache for quick access by the processor 4520. In this way, the cache provides a performance boost that avoids processor 4520 delays while waiting for data. These and other modules can control or be configured to control the processor 4520 to perform various actions. Other system memory 4530 may be available for use as well. The memory 4530 can include multiple different types of memory with different performance characteristics. It can be appreciated that the disclosure may operate on a computing device 4500 with more than one processor 4520 or on a group or cluster of computing devices networked together to provide greater processing capability. The processor 4520 can include any general purpose processor and a hardware module or software module, such as module 1 4562, module 2 4564, and module 3 4566 stored in storage device 4560, configured to control the processor 4520 as well as a special-purpose processor where software instructions are incorporated into the actual processor design. The processor 4520 may essentially be a completely self-contained computing system, containing multiple cores or processors, a bus, memory controller, cache, etc. A multi-core processor may be symmetric or asymmetric.

The system bus 4510 may be any of several types of bus structures including a memory bus or memory controller, a peripheral bus, and a local bus using any of a variety of bus architectures. A basic input/output (BIOS) stored in ROM 4540 or the like, may provide the basic routine that helps to transfer information between elements within the computing device 4500, such as during start-up. The computing device 4500 further includes storage devices 4560 such as a hard disk drive, a magnetic disk drive, an optical disk drive, tape drive or the like. The storage device 4560 can include software modules 4562, 4564, 4566 for controlling the processor 4520. Other hardware or software modules are contemplated. The storage device 4560 is connected to the system bus 4510 by a drive interface. The drives and the associated computer-readable storage media provide nonvolatile storage of computer-readable instructions, data structures, program modules and other data for the computing device 4500. In one aspect, a hardware module that performs a particular function includes the software component stored in a tangible computer-readable storage medium in connection with the necessary hardware components, such as the processor 4520, bus 4510, display 470, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 4500 is a small, handheld computing device, a desktop computer, or a computer server.

Although the exemplary embodiment described herein employs the hard disk 4560, other types of computer-readable media which can store data that are accessible by a computer, such as magnetic cassettes, flash memory cards, digital versatile disks, cartridges, random access memories (RAMs) 4550, and read only memory (ROM) 4540, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 4500, an input device 4590 represents any number of input mechanisms, such as a microphone for speech, a touch-sensitive screen for gesture or graphical input, keyboard, mouse, motion input, speech and so forth. An output device 4570 can also be one or more of a number of output mechanisms known to those of skill in the art. In some instances, multimodal systems enable a user to provide multiple types of input to communicate with the computing device 4500. The communications interface 480 generally governs and manages the user input and system output. There is no restriction on operating on any particular hardware arrangement and therefore the basic features here may easily be substituted for improved hardware or firmware arrangements as they are developed.

The above mentioned and described embodiments are only given as examples and should not be seen to be limiting to the present invention. Other solutions, uses, objectives, and functions within the scope of the invention as claimed in the below described patent claims should be apparent for the person skilled in the art.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic.

Further, repeated use of the phrase “in one embodiment,” or “in an illustrative embodiment,” do not necessarily refer to the same embodiment, although they may. The various embodiments described herein may be combined and/or features of the embodiments may be combined to form new embodiments.

Unless specifically stated otherwise, as apparent from the following discussions, it is appreciated that throughout the specification discussions utilizing terms such as “processing,” “computing,” “calculating, ” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

In a similar manner, the term “processor” may refer to any device or portion of a device that processes electronic data from registers and/or memory to transform that electronic data into other electronic data that may be stored in registers and/or memory. A “computing platform” may comprise one or more processors and/or databases.

Embodiments of the invention may include apparatuses for performing the operations herein. An apparatus may be specially constructed for the desired purposes, or it may comprise a general purpose device selectively activated or reconfigured by a program stored in the device.

Embodiments may be embodied in many different ways as a software component. For example, it may be a stand-alone software package, or it may be a software package incorporated as a “tool” in a larger software product, such as, for example, a scientific modeling product. It may be downloadable from a network, for example, a website, as a stand-alone product or as an add-in package for installation in an existing software application. It may also be available as a client-server software application, or as a web-enabled software application. One or more computers may be specialized by storing programming logic that enables one or more processors to perform the techniques indicated herein.

While various embodiments of the invention have been described above, it should be understood that they have been presented by way of example only, and not limitation. Thus, the breadth and scope of the invention should not be limited by any of the above-described illustrative embodiments, but should instead be defined only in accordance with the following claims and their equivalents. The embodiments of the invention that have been described above may contain features that may be removed or combined between the described embodiments to derive additional embodiments.

What is claimed is:

1. A method for telemedicine referral implemented on a computer, said computer comprising a server and a data storage device in communication with said server, said data storage device comprising a database of information for each of a plurality of dermatologists stored thereon, said method comprising:
receiving, by said server, a case information package from a patient-facing computing device, the case information package including at least one image of a skin condition of a patient;
selecting, by the server, at least one dermatologist from the plurality of dermatologists to be provided with at least a portion of the case information package;
transmitting, by the server, at least a portion of the case information package to a dermatologist-facing computing device corresponding to each of the selected at least one dermatologist;
receiving, by the server, from the dermatologist-facing computing device, assessment information of the skin condition, the assessment information being determined by the selected at least one dermatologist based on, at least, the at least said portion of the case information package; and
transmitting, by the server, the assessment information to the patient-facing computing device.

2. The method of claim 1, further comprising:
processing, by the server, the case information package, the processing including classifying the skin condition as a specific type of skin condition.

3. The method of claim 2, wherein the selecting, by the server, at least one dermatologist, is based on, at least, the classification of the skin condition.

4. The method of claim 2, wherein the classification of the skin condition is performed via image recognition techniques.

5. The method of claim 1, wherein the assessment information includes: a name of said selected at least one dermatologist who diagnosed the skin condition, a date on which the assessment information was submitted by said selected at least one dermatologist, a date on which the skin condition was diagnosed by said selected at least one dermatologist, and medical advice on how to remedy the skin condition.

6. The method of claim 1, wherein the at least one image of the skin condition is a clinical image of the skin condition or a dermascopic image of the skin condition.

7. The method of claim 1, further comprising:
organizing the case information package in a queue according to a priority of the case information package;
updating the priority based on at least one of: a rank of a dermatologist who rejects the case information package, a number of times the case information package has been rejected, and a period of time the case information package is held on the server.

8. The method of claim 1, further comprising:
providing a list of potential conditions of the skin condition to the selected at least one dermatologist for selecting a potential condition for the skin condition;
storing a list of preferred diagnoses of the selected at least one dermatologist;
generating a list of cases completed by the selected at least one dermatologist; and
generating a diagnosis statistics for the selected at least one dermatologist.

9. The method of claim 1, further comprising:
receiving a—payment from the patient-facing computing device.

10. The method of claim 1, further comprising:
generating a case identifier for the case information package; and
sending the case number anonymously to the dermatologist-facing computing device.

11. A system for telemedicine referral, comprising:
a server; and
a data storage device in communication with said server, said data storage device comprising a database of information for each of a plurality of dermatologists stored thereon,
wherein said server is configured to:
receive a case information package from a patient-facing computing device, the case information package including at least one image of a skin condition of a patient;
select at least one dermatologist from the plurality of dermatologists to be provided with at least a portion of the case information package;
transmit at least a portion of the case information package to a dermatologist-facing computing device corresponding to each of the selected at least one dermatologist;
receive from the dermatologist-facing computing device, assessment information of the skin condition, the assessment information being determined by the selected at least one dermatologist based on, at least, the at least said portion of the case information package; and
transmit the assessment information to the patient-facing computing device.

12. The system of claim 11, wherein said server is further configured to:
process the case information package, the processing including classifying the skin condition as a specific type of skin condition.

13. The system of claim 12, wherein said server is further configured to select at least one dermatologist, based on, at least, the classification of the skin condition.

14. The system of claim 12, wherein the classification of the skin condition is performed via image recognition techniques.

15. The system of claim 11, wherein said server is further configured to:
organize the case information package in a queue according to a priority of the case information package;
update the priority based on at least one of: a rank of a dermatologist who rejects the case information package, a number of times the case information package has been rejected, and a period of time the case information package is held on the server.

16. The system of claim 11, wherein said server is further configured to:
provide a list of potential conditions of the skin condition to the selected at least one dermatologist for selecting a potential condition for the skin condition;
store a list of favorite diagnoses of the selected at least one dermatologist;
generate a list of cases completed by the selected at least one dermatologist; and
generate a diagnosis statistics for the selected at least one dermatologist.

17. A non-transitory computer-readable medium having instructions stored thereon that, when executed, cause a computer to perform a method for telemedicine referral, said computer comprising a server and a data storage device in communication with said server, said data storage device comprising a database of information for each of a plurality of dermatologists stored thereon, said method comprising:
receiving, by said server, a case information package from a patient-facing computing device, the case information package including at least one image of a skin condition of a patient;
selecting, by the server, at least one dermatologist from the plurality of dermatologists to be provided with at least a portion of the case information package;
transmitting, by the server, at least a portion of the case information package to a dermatologist-facing computing device corresponding to each of the selected at least one dermatologist;
receiving, by the server, from the dermatologist-facing computing device, assessment information of the skin condition, the assessment information being determined by the selected at least one dermatologist based on, at least, the at least said portion of the case information package; and
transmitting, by the server, the assessment information to the patient-facing computing device.

18. The non-transitory computer-readable medium of claim 17, wherein said method further comprises:
processing, by the server, the case information package, the processing including predicting classifying the skin condition as a specific type of skin condition.

19. The non-transitory computer-readable medium of claim 18, wherein the selecting at least one dermatologist, is based on, at least, the classification of the skin condition.

20. The non-transitory computer-readable medium of claim 18, wherein the classification of the skin condition is performed via image recognition techniques.
